(19) United States
(12) Patent Application Publication
(10) Pub. No.: US 2014/0135790 A1
(43) Pub. Date: May 15, 2014

(54) SYSTEM AND METHOD FOR GUIDING A MEDICAL DEVICE TO A TARGET REGION

(71) Applicants: Aaron Fenster, London (CA); Jeremy Cepek, Wyoming (CA); Jeffrey Bax, London (CA); Uri Lindner, Toronto (CA); John Trachtenberg, Toronto (CA)

(72) Inventors: Aaron Fenster, London (CA); Jeremy Cepek, Wyoming (CA); Jeffrey Bax, London (CA); Uri Lindner, Toronto (CA); John Trachtenberg, Toronto (CA)

(21) Appl. No.: 14/043,286

(22) Filed: Oct. 1, 2013

Related U.S. Application Data
(60) Provisional application No. 61/708,636, filed on Oct. 1, 2012.

(51) Int. Cl. A61B 19/00

(52) U.S. Cl. CPC A61B 19/201
USPC 606/130

(57) ABSTRACT

A device guiding apparatus comprises support framework, a counterbalance supported by the support framework at a position above a surface on which the support framework rests, and a manipulation assembly supported by the counterbalance. The manipulation assembly comprises at least one support assembly for supporting a medical device at a position intermediate the counterbalance and the surface such that a user has a direct line-of-site of the at least one support assembly.

### CROSS-REFERENCE TO RELATED APPLICATIONS

This application claims the benefit of U.S. Provisional Application No. 61/708,636 to Cepek et al. filed on Oct. 1, 2012, the entire content of which is incorporated herein by reference.

### FIELD OF THE INVENTION

The present invention relates to a system and method for guiding a medical device to a target region.

### BACKGROUND OF THE INVENTION

Magnetic resonance (MR) imaging has been recognized as an extremely versatile medical imaging modality that has many applications. For example, MR imaging can be used to visualize prostate cancer, to visualize needles during insertion, and to visualize temperature during thermal therapies. As such, several researchers and clinicians have investigated the feasibility of using MR imaging for delivering focal therapy to patients with prostate cancer. For therapies requiring guidance of a needle, methods have been developed, such as focal therapy, focus laser ablation (FLA), etc. which require the insertion of needles through the patient's perineum. These methods are typically performed with the patient positioned in the semi-prone position within the bore of an MR imaging scanner. As will be appreciated, this position minimizes patient motion during imaging while maximizing patient comfort, both of which are important factors in a procedure that can last for several hours.

FLA is performed by inserting an open-ended or translucent catheter into the prostate through the patient's perineum. An optical fiber with a diffusing tip is inserted through the catheter to the tumor site, and is attached to a laser for thermal ablation (1).

MR imaging-guided FLA of prostate cancer has been tested. It was found that MR imaging provided excellent visualization of the needle for guidance, thermal monitoring and damage estimation during the ablation using MR thermometry, and intraoperative visualization of the ablated region.

While MR imaging provides a full suite of tools for MR imaging-guided FLA, a method and system for accurately guiding the therapy to the tumor site are desired. Further, the accuracy of FLA methods must be evaluated in vivo to enable evaluation of the potential clinical efficacy of prostate cancer focal therapies.

The use of MR imaging for guiding therapy or biopsies has resulted in the development of various systems (2 to 8). While these systems have shown promise with respect to targeting accuracy, issues remain regarding reductions in image signal-to-noise ratio (SNR), procedure workflow, and patient safety.

SNR reduction is caused by the use of electromechanical actuators that increase the noise in the MR imaging scanners' radio frequency (RF) receive coils, especially if the actuators are moved during imaging (3, 5, 9).

The main obstacle with respect to procedure workflow is due to the limited workspace around the patient when the patient is positioned within the bore of the MR imaging scanner, and due to the fact that the patient's prostate is generally one (1) meter into the MR imaging scanner bore. The general solution to this problem has been to remove the patient from the MR imaging scanner bore for needle insertion, and then move the patient back into the MR imaging scanner bore for verification of needle depth with MR imaging (4 to 6, 8). As will be appreciated, since the needle cannot be visualized while it is being inserted, this method requires incremental insertions, with multiple translations of the patient into and out of the MR imaging scanner bore. Moving the patient into and out of the MR imaging scanner bore results in excessive movement, reducing potential accuracy and increasing procedure time.

Systems (3, 5) have been developed that are fully automated, however patient safety may be compromised since there is no haptic feedback or safety system in place.

As will be appreciated, improvements are generally desired. It is therefore an object at least to provide a novel system and method for guiding a medical device to a target region.

### SUMMARY OF THE INVENTION

Accordingly, in one aspect there is provided a device guiding apparatus, comprising support framework, a counterbalance supported by the support framework at a position above a surface on which the support framework rests, and a manipulation assembly supported by the counterbalance, the manipulation assembly comprising at least one support assembly for supporting a medical device at a position intermediate the counterbalance and the surface such that a user has a direct line-of-site of the at least one support assembly.

In an embodiment, the support framework, the manipulation assembly, the at least one support assembly and the counterbalance are made of non-magnetic materials. In an embodiment, the device guiding apparatus is positionable within a bore of an MR imaging scanner. In an embodiment, the device guiding apparatus comprises a sensor arrangement for determining the trajectory of the medical device. In an embodiment, the device guiding apparatus comprises an alignment interface providing feedback to the user for adjusting the orientation of the medical device.

According to another aspect there is provided a device guiding apparatus, comprising support framework, a counterbalance supported by the support framework at a position above a surface on which the support framework rests, and a manipulation assembly supported by the counterbalance, the manipulation assembly comprising at least one support assembly for supporting a medical device at a position intermediate the counterbalance and the surface such that a user has a direct line-of-site of the at least one support assembly, a sensor arrangement configured to obtain sensor data, and processing structure configured to receive sensor data from the sensor arrangement, process the received sensor data to determine the trajectory of the medical device, calculate a point of intersection with a target region based on the trajectory of the medical device, calculate a difference between the point of intersection and a target point associated with the target region, and provide feedback to the user to guide the medical device to the target point based on said calculated difference.

According to another aspect there is provided a method for providing feedback to a user guiding a medical device to a target region, the method comprising receiving sensor data from a sensor arrangement, processing the received sensor data to determine the trajectory of the medical device, calculating a point of intersection with a target region based on the trajectory of the medical device, calculating a difference between the point of intersection and a target point associated with the target region, and providing feedback to the user based on the calculated difference between the point of intersection and the target point.

According to another aspect there is provided a non-transitory computer readable medium having stored thereon a computer program comprising computer readable instructions for execution by a computer to perform a method of providing feedback to a user guiding a medical device to a target region, the method comprising receiving sensor data from a sensor arrangement, processing the received sensor data to determine the trajectory of the medical device, calculating a point of intersection with a target region based on the trajectory of the medical device, calculating a difference between the point of intersection and an actual target point associated with the target region, and providing feedback to the user based on the calculated difference between the estimated target point and the actual target point.

### BRIEF DESCRIPTION OF THE DRAWINGS

Embodiments will now be described more fully with reference to the accompanying drawings in which:

FIG. 1 is schematic block diagram of a system for guiding a medical device to a target region;

FIG. 2 is an isometric view of a device guiding apparatus forming part of the system of FIG. 1;

FIG. 3 is an isometric view of a frame forming part of the device guiding apparatus of FIG. 2;

FIGS. 4a to 4c are isometric, bottom and side views, respectively, of a linear motion assembly forming part of the device guiding apparatus of FIG. 2;

FIG. 5 is a side view of an extension assembly forming part of the device guiding apparatus of FIG. 2;

FIG. 6 is a side view of a manipulation assembly forming part of the device guiding apparatus of FIG. 2;

FIG. 7 is an isometric view of a counterbalance assembly forming part of the device guiding apparatus of FIG. 2;

FIG. 8 is a cross-sectional view of a locking assembly forming part of the device guiding apparatus of FIG. 2;

FIG. 9 is an isometric view of a sensor arrangement forming part of the device guiding apparatus of FIG. 2;

FIGS. 10a to 10c show views of an alignment interface forming part of the system of FIG. 1;

FIGS. 11 and 12 show the coordinates associated with the device guiding apparatus of FIG. 2;

FIG. 13 shows a detachable fiducial MR-visible component; and

FIGS. 14a and 14b show exemplary MR images of the fiducial MR-visible components of FIG. 13.

### DETAILED DESCRIPTION OF THE EMBODIMENTS

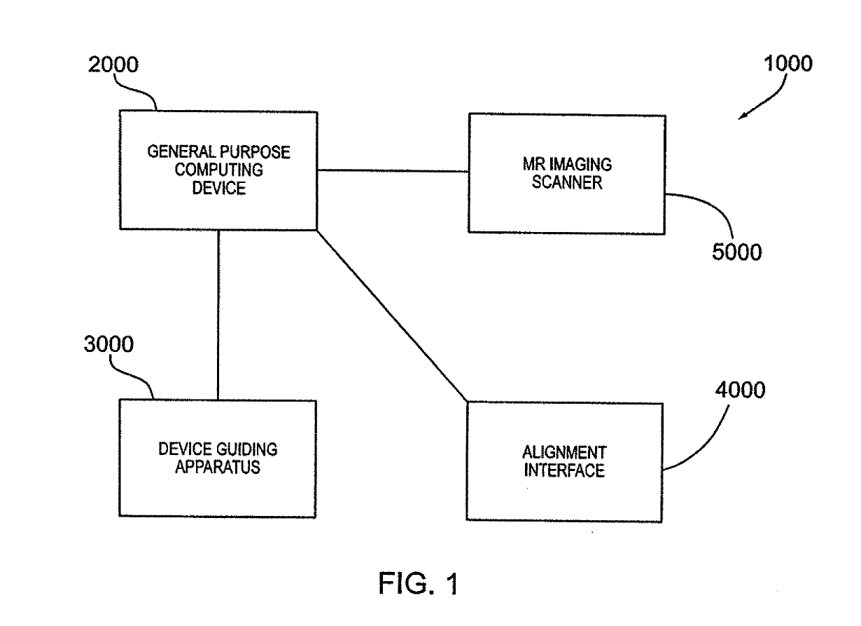

Turning to FIG. 1, a system for guiding a medical device to a target region is shown and is generally identified by reference numeral 1000. In this example, the medical device is a needle and the target region is a patient's prostate. The system 1000 comprises a general purpose computing device 2000 that is communicatively coupled to a device guiding apparatus 3000, an alignment interface 4000, and a magnetic resonance (MR) imaging scanner 5000. In this embodiment, the device guiding apparatus 3000 is positioned within the bore of the MR imaging scanner 5000. The device guiding apparatus 3000 is operable in two modes: target only mode and target and entry mode, as will be described below. The general purpose computing device 2000 communicates with the MR imaging scanner 5000 via a file transfer protocol and receives MR images of a target region therefrom. The MR images are processed to register the orientation of a medical device, such as for example a needle, supported by the device guiding apparatus 3000, to select a target point associated with the target region, and to monitor the medical device during use. The general purpose computing device 2000 communicates with the device guiding apparatus 3000 to determine the precise location and orientation of the medical device. The general purpose computing device 2000 compares the location and orientation of the medical device to that required to reach the target point. The general purpose computing device 2000 in turn provides output to the alignment interface 4000 to provide feedback to the physician to enable the physician to adjust the location and orientation of the medical device using the device guiding apparatus 3000, if required.

The general purpose computing device 2000 in this embodiment is a personal computer or other suitable processing device comprising, for example, a processing unit, system memory (volatile and/or non-volatile memory), other non-removable or removable memory (e.g., a hard disk drive, RAM, ROM, EEPROM, CD-ROM, DVD, flash memory, etc.) and a system bus coupling the various computing device components to the processing unit. The general purpose computing device 2000 may also comprise networking capability using Ethernet, WiFi, and/or other network formats, to access shared or remote drives, one or more networked computers, or other networked devices.

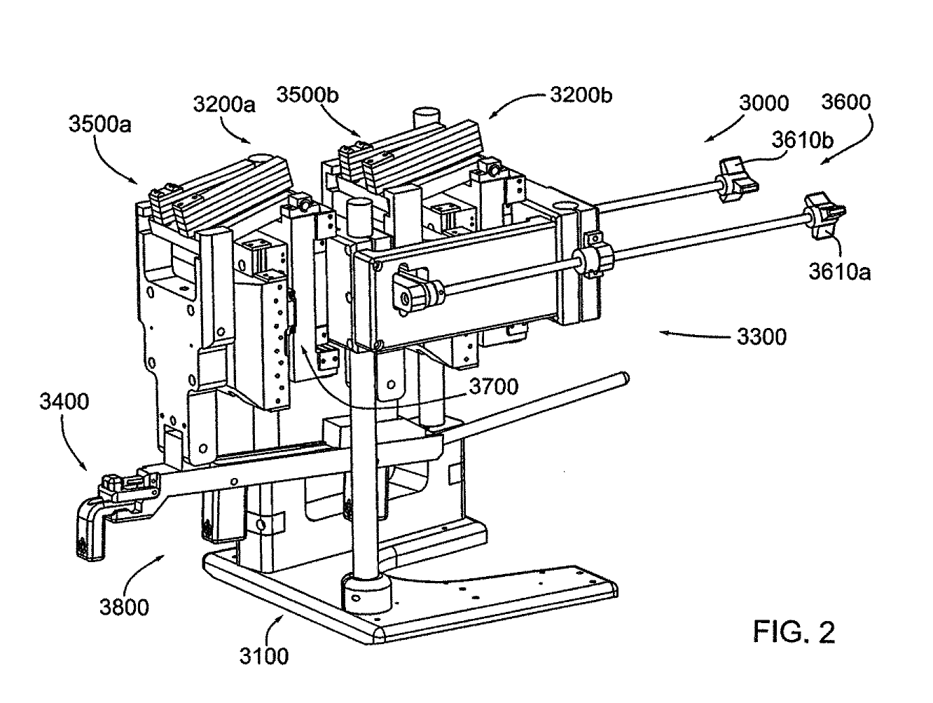

FIGS. 2 to 9 illustrate the device guiding apparatus 3000. In this embodiment, as the device guiding apparatus 3000 is positioned within the bore of the MR imaging scanner 5000, all components of the device guiding apparatus 3000 are made of non-magnetic material. As can be seen, the device guiding apparatus 3000 comprises a frame 3100, a pair of linear motion assemblies 3200a and 3200b, an extension assembly 3300, a manipulation assembly 3400, a counterbalance comprising a pair of counterbalance assemblies 3500a and 3500b, each of which is associated with a respective one of the linear motion assemblies 3200a and 3200b, a locking assembly 3600 and a sensor arrangement 3700, the specifics of which will now be described.

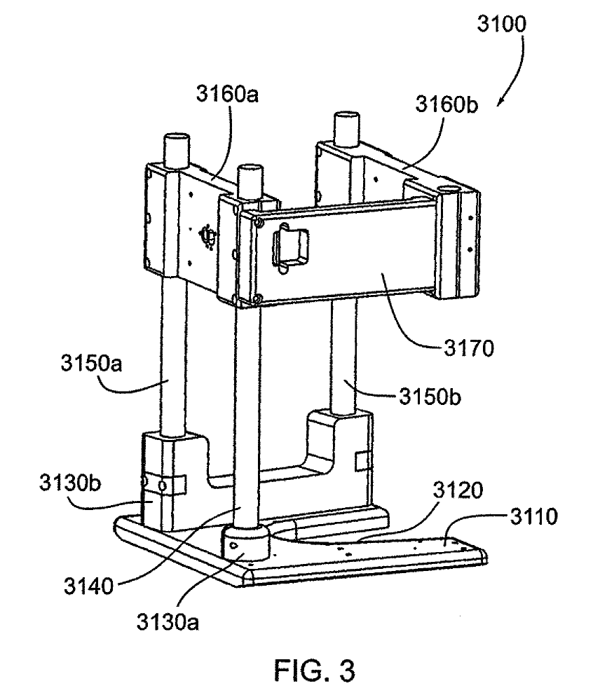

FIG. 3 better illustrates the frame 3100. As can be seen, the frame 3100 comprises a base 3110 made of a generally flat sheet of plastic material, such as for example Delrin®, having a U-shaped cut out 3120 therein. The cut out 3120 increases the amount of clearance available for a physician's hand while manipulating a medical device supported by the device guiding apparatus 3000. Lower brackets 3130a and 3130b are connected to the base 3110. Each of the lower brackets 3130a and 3130b is made of plastic material, such as for example Delrin®. Lower bracket 3130a is connected to an upwardly extending pillar 3140 via a shaft clamp assembly (not shown). Lower bracket 3130b is connected to a pair of upwardly extending pillars 3150a and 3150b via shaft clamp assemblies (not shown). Each of the pillars 3140, 3150a and 3150b is made of a non-metallic fiberglass material. As will be further described below, the pillars 3140, 3150a and 3150b provide elevation to components of the device guiding apparatus 3000 to increase the amount of space available for the physician. An upper bracket 3160a is connected to pillars 3140 and 3150a via shaft clamp assemblies (not shown) adjacent its opposite ends and an upper bracket 3160b is connected adjacent one of its ends to the pillar 3150b via a clamp shaft assembly (not shown). Each of the upper brackets 3160a and 3160b is made of a plastic material, such as for example Delrin®. A support brace 3170 extends between the upper brackets 3160a and 3160b and is made of a plastic material, such as for example Delrin®. The support brace 3170 is used to reduce the number of pillars required to support the components of the device guiding apparatus 3000 and thereby further increases the amount of space available to the physician. The upper brackets 3160a and 3160b and support brace 3170 are dimensioned to receive the linear motion assemblies 3200a and 3200b, as will now be described.

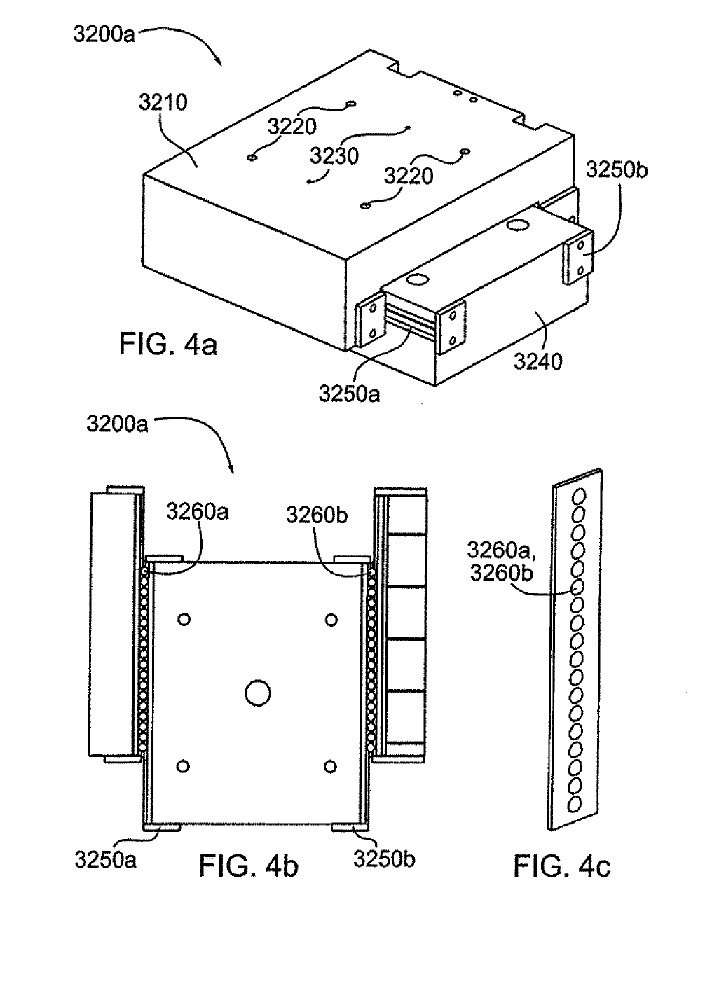

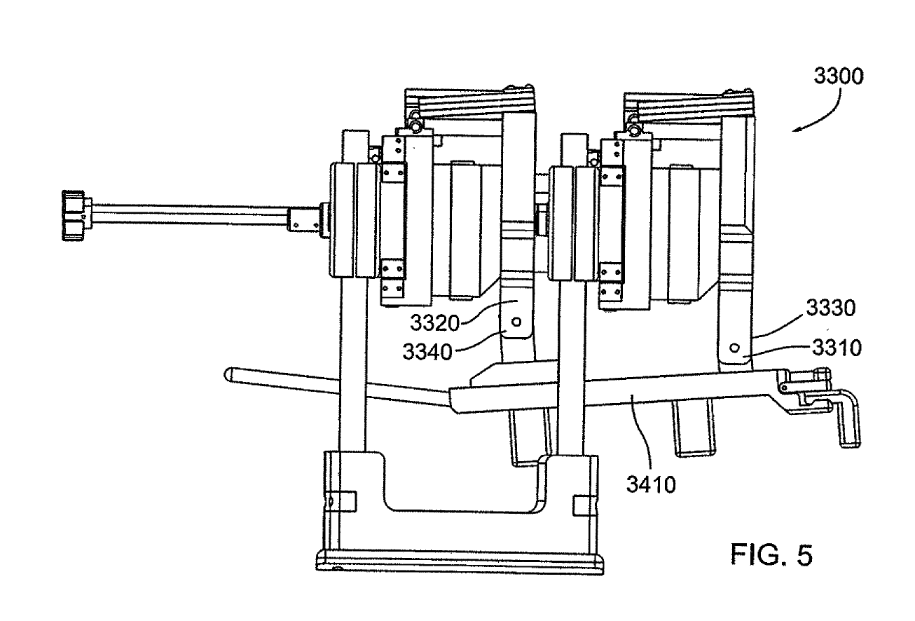

FIGS. 4a to 4c illustrate the linear motion assembly 3200a. As the linear motion assemblies 3200a and 3200b are similar, only linear motion assembly 3200a will be described. Linear motion assembly 3200a is dual-axis and comprises two stages, an x-stage and a y-stage, connected to one another at an angle of ninety (90) degrees. Each stage comprises a carriage 3210 having four (4) mounting holes 3220 and two (2) locating holes 3230 therein. The carriage 3210 is made of a plastic material, such as for example Delrin®. The carriage 3210 is connected to a rail 3240 made of a plastic material, such as for example Delrin®. The rail 3240 is connected to bearing races 3250a and 3250b such that each bearing race 3250a and 3250b extends along one side of the rail 3240. Ball bearing assemblies 3260a and 3260b are fixably connected to the carriage 3210 and each receives one of the bearing races 3250a and 3250b such that the carriage 3210 can move along a single axis with respect to the rail 3240. In this embodiment, the ball bearing assemblies 3260a and 3260b are made of a non-magnetic material. As mentioned previously, the linear motion assembly 3200a is connected to the upper brackets 3160a and 3160b and support brace 3170 of the frame 3100.

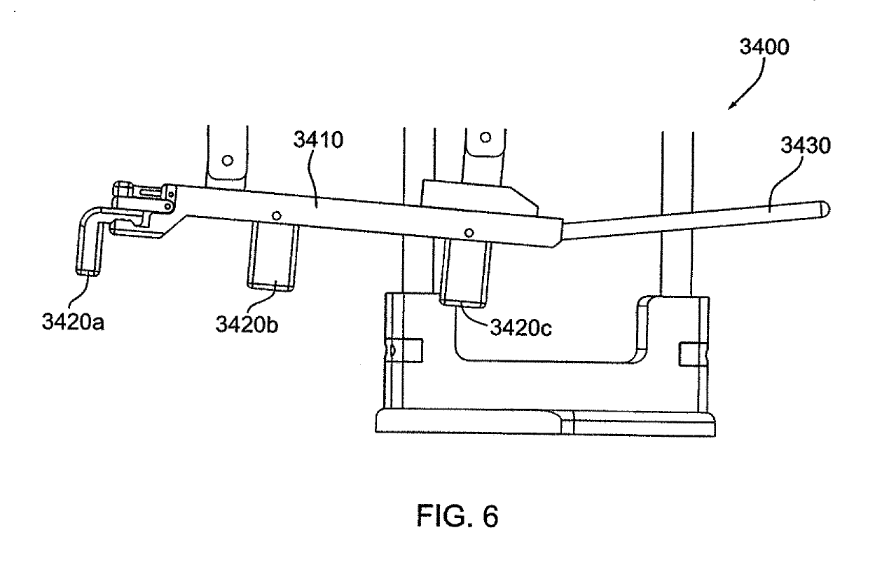

Turning now to FIGS. 5 and 6, the extension assembly 3300 and the manipulation assembly 3400 are better shown. The extension assembly 3300 comprises a front extension arm 3310 and a rear extension arm 3320. A first end of the front extension arm 3310 is connected to a spring balance arm of the counterbalance assembly 3500a. The body of the front extension arm 3310 is connected to the carriage of the y-stage linear motion assembly 3200a. A second end of the front extension arm 3310 is connected to a manipulator arm 3410 of the manipulation assembly 3400 (described below) via a spherical joint 3330. A first end of the rear extension arm 3320 is connected to a spring balance arm of the counterbalance assembly 3500b. The body of the rear extension arm 3320 is connected to the y-stage of the linear motion assembly 3200b. A second end of the rear extension arm 3320 is connected to the manipulator arm 3410 of the manipulation assembly 3400 via a spherical joint 3340. Each spherical joint 3330 and 3340 provides the manipulation assembly 3400 with two rotational degrees-of-freedom. As will be appreciated, the extension assembly 3300 allows manipulation assembly 3400 to follow the motion of each linear motion assembly 3200a and 3200b.

In this embodiment the manipulator arm 3410 is made of a plastic material, such as for example Delrin®. The manipulator arm 3410 has one or more support assemblies for supporting the medical device thereon. In this embodiment, the support assemblies are three (3) needle templates 3420a, 3420b and 3420c mounted on the manipulator arm 3410 at spaced locations. Each of the needle templates 3420a to 3420c is made of a plastic material, such as for example polyether ether ketone (PEEK). An alignment handle 3430 is connected to the rearward end of the manipulator arm 3410 and extends therefrom. The alignment handle 3430 allows the physician to manually manipulate the position and orientation of the manipulator arm 3410 thereby adjusting the position and orientation of the medical device supported by the needle templates 3420a, 3420b and 3420c. In this embodiment the alignment handle 3430 is made of a plastic material, such as for example PEEK. The rear needle template 3420c is used as an extension of the middle needle template 3420b and front needle template 3420a. In this embodiment, the rear needle template 3420c allows the physician to guide a needle into the patient from outside the MR imaging scanner bore. Thus, a direct line-of-site of the rear needle template 3420c is provided allowing the physician to guide the needle through the rear needle template 3420c, middle needle template 3420b and front needle template 3420a, towards the patient.

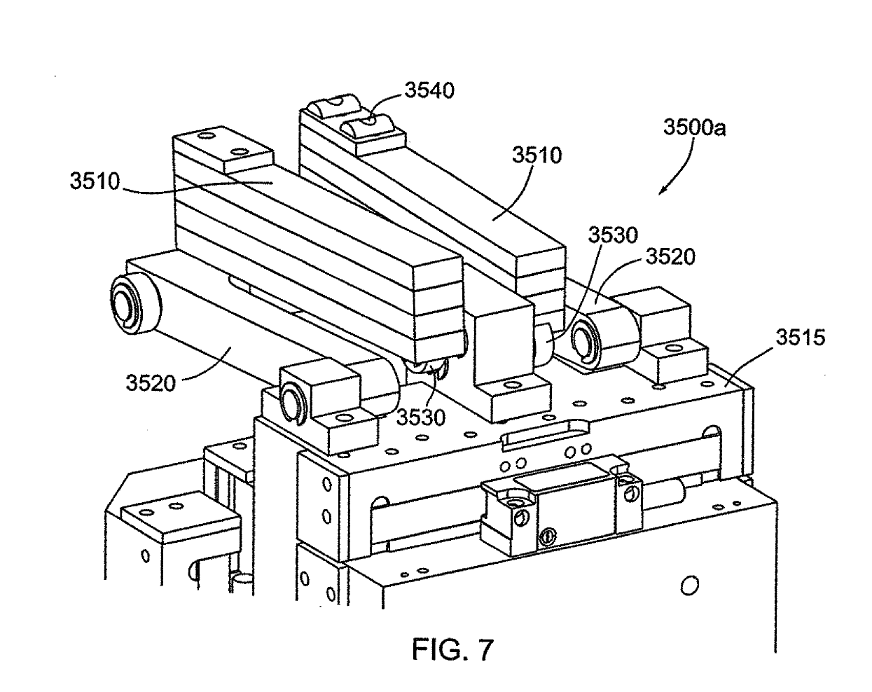

Turning now to FIG. 7, the counterbalance assembly 3500a is shown. As the counterbalance assemblies 3500a and 3500b are similar, only counterbalance assembly 3500a will be described. In this embodiment, the counterbalance assembly 3500a is similar to that described in U.S. Patent Application Publication No. 2010/0319164 to Bax et al., the relevant portions of the disclosure of which are incorporated herein by reference. The counterbalance assembly 3500a is used to allow components of the device guiding apparatus 3000 coupled thereto and having a vertical degree-of-freedom to remain in the position placed by the physician without the force of gravity moving them downward. As will be appreciated, this allows the physician to adjust each degree-of-freedom of the device guiding apparatus 3000 using one hand on the alignment handle 3430. In this embodiment, the counterbalance assembly 3500a comprises two sets of four (4) biasing elements in the form of leaf springs 3510, or other suitable spring-like arrangements. Each of the leaf springs is made of a plastic material such as for example PEEK. A first end of each set of the leaf springs 3510 is connected to a support arm 3515. A second end of each of the set of leaf springs 3510 is connected to a U-shaped spring balance arm 3520. The spring balance arm 3520 provides mounting for the leaf springs 3510 and ensures that the counterbalance assembly 3500a has a single degree-of-freedom and transfers the vertical force of the counterbalance assembly 3500a to the extension arm connected thereto. As will be appreciated, the spring balance aim 3520 must be able to support the torque of the leaf springs 3510, and thus is made of a stiffer material than the leaf springs 3510. In this embodiment, the spring balance arm 3520 is made of aluminum, which is generally stiffer than PEEK.

The counterbalance assembly 3500a also comprises two (2) cam bearings 3530. The cam bearings 3530 are offset from the rotational axis of the spring balance arm 3520, and are offset ninety (90) degrees from one another. As a result, the counterbalance assembly 3500a provides the force for offsetting the force of gravity, and compensates for the component of the force that varies with the position of the medical device. The arrangement of the cam bearings 3530 provides a constant force independent of the position of the medical device. In this embodiment, the cam bearings 3530 are made of a ceramic material.

An adjustment screw (not shown) may be used with adjustment screw hole 3540 to adjust the tension of the leaf springs 3510.

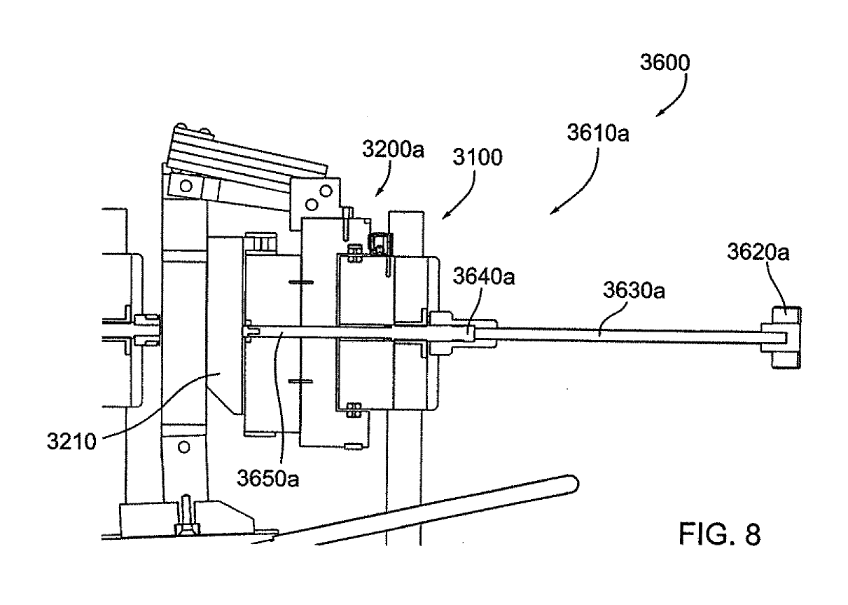

Referring back to FIG. 2, the locking assembly 3600 comprises a front locking assembly 3610a and a rear locking assembly 3610b. The front locking assembly 3610a is shown in FIG. 8 and comprises a locking handle 3620a connected to a locking shaft 3630a. The locking shaft 3630a has a large diameter section 3640a that is received through an opening in the frame 3100 via a threaded connection. The locking shaft 3630a has a small diameter section 3650a that extends through linear motion assembly 3200a and is moveable relative to the carriage 3210. When in an unlocked position, the locking shaft 3630a is not in contact with the carriage 3210. When the locking handle 3620a is turned clockwise, the locking shaft 3630a advances into contact with the carriage 3210 of the y-stage so that it assumes the locked position. In the locked position, the locking assembly 3610a prevents motion of the linear motion assembly 3200a due to the frictional force between the locking shaft 3630a and the carriage 3210. A pair of mechanical stops (not shown) inhibits over-tightening or over-loosening of the locking assembly 3600. The rear locking assembly 3610b is similar to the front locking assembly 3610a and contacts the carriage 3210 associated with y-stage of the linear motion assembly 3200b when in the locked position to inhibit it from moving.

When both locking assemblies 3610a and 3610b are unlocked, both the linear motion assemblies 3200a and 3200b are allowed to move in response to manipulation of the manipulator arm 3410 via the alignment handle 3420. As such, the needle templates 3420a, 3420b and 3420c may be rotated and/or translated about all four rotational degrees-of-freedom (defined by spherical joints 3330 and 3340) via the manipulator arm 3410 thereby adjusting the orientation of the medical device.

When locking assembly 3610a is locked and locking assembly 3610b is unlocked, only the linear motion assembly 3200b is allowed to move in response to manipulation of the manipulator arm 3410 via the alignment handle 3420. As such, the angle of the needle templates 3420a, 3420b and 3420c may be manipulated about the spherical joint 3340 via the manipulator arm 3410 thereby adjusting the angle of the medical device.

When locking assembly 3610a is unlocked and locking assembly 3610b is locked, only the linear motion assembly 3200a is allowed to move in response to manipulation of the manipulator arm 3410 via the alignment handle 3420. As such, the angle of the needle template 3420a may be manipulated about the spherical joint 3330 via the manipulator arm 3410 thereby adjusting the angle of the medical device.

When both locking assemblies 3610a and 3610b are locked, neither of the linear motion assemblies 3200a and 3200b are allowed to move in response to manipulation of the manipulator arm 3410 via the alignment handle 3420. As such, the needle templates 3420a, 3420b and 3420c are unable to move and the medical device remains stationary.

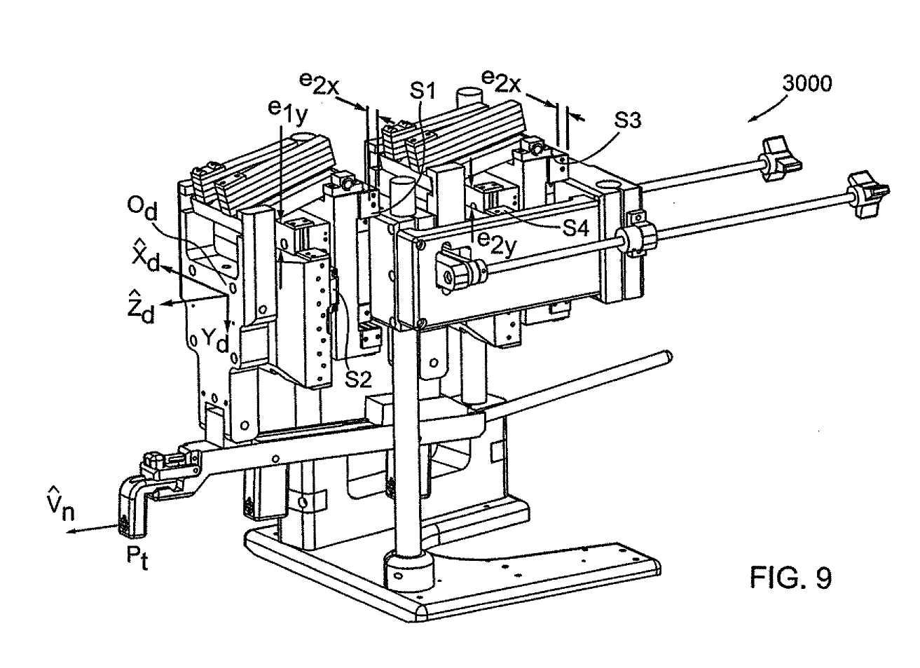

Turning now to FIG. 9, the sensor arrangement 3700 is better shown. As can be seen, the sensor arrangement 3700 comprises four (4) encoders S1 to S4. In this embodiment, the encoders S1 to S4 are magnetic rotary encoders, such as the MR-compatible linear optical encoders LIA-20 manufactured by Numerik Jena, and used to measure the angle between the encoder body (identified as S1 to S4) and an associated encoder magnet (not shown). Each of the encoders S1 to S4 is connected to the general purpose computing device 2000 via a wired connection (not shown). An RF filter (not shown) is used to remove noise introduced into the wired connections. The encoders S1 to S4 are constructed of non-magnetic materials and output a sine-cosine signal in the kHz range. Encoder S1 is positioned to measure the x-component (e₁ₓ) of the linear motion assembly 3200a. Encoder S2 is positioned to measure the y-component (e_{1y}) of the linear motion assembly 3200a. Encoder S3 is positioned to measure the x-component (e₂ₓ) of the linear motion assembly 3200b. Encoder S4 is positioned to measure the y-component (e_{2y}) of the linear motion assembly 3200b.

The device guiding apparatus 3000 coordinate system is also shown in FIG. 9 and is defined as (X_{d}, Y_{d}, Z_{d}) at origin O_{d}. The needle trajectory, represented by point pₜ and needle vector \hat{v}ₙ, is calculated by the general purpose computing device 2000 using the (x, y) coordinates of linear motion assemblies 3200a and 3200b. The (x,y) coordinates of linear motion assemblies 3200a and 3200b are measured by encoders S1 to S4 as coordinates (e₁ₓ, e_{1y}) and (e₂ₓ, e_{2y}), respectively.

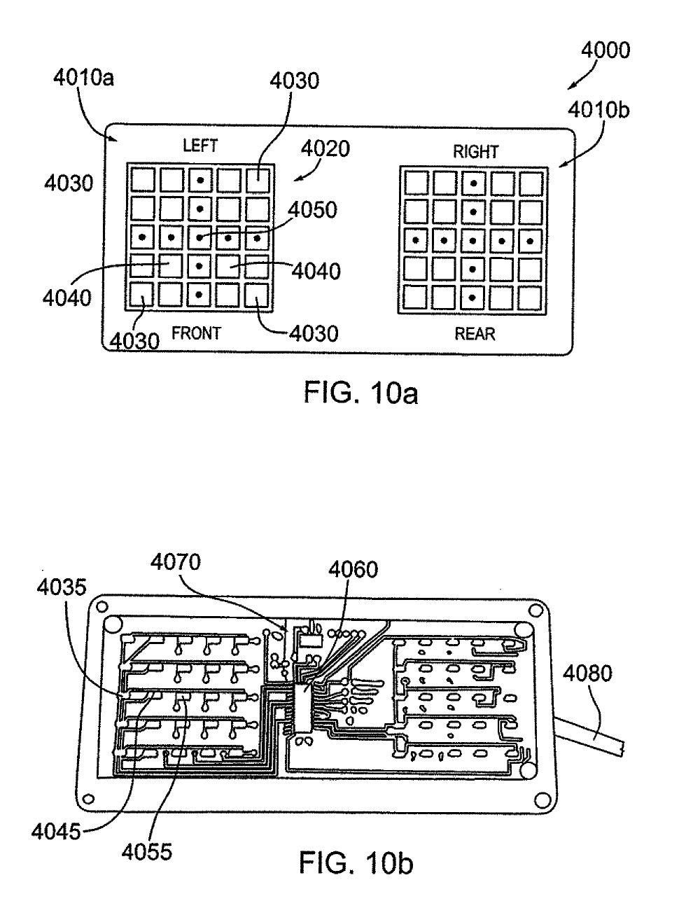

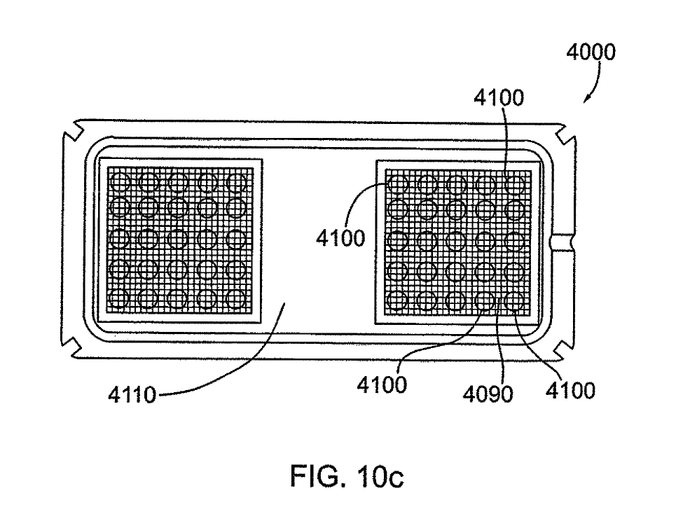

Turning now to FIGS. 10a to 10c, the alignment interface 4000 is shown. The alignment interface 4000 allows the physician to align the medical device with the target point associated with the target region, which in this embodiment is selected in an MR image. The alignment interface 4000 permits manual control of the device guiding apparatus 3000 while providing immediate haptic feedback and ensuring patient safety. In this embodiment, the alignment interface 4000 is MR compatible and thus can be placed adjacent to the device guiding apparatus 3000 either inside or outside of the bore of the MR imaging scanner 5000. As will be described, the alignment interface 4000 helps the physician guide the medical device to the target point associated with the target region by representing varying levels of accuracy.

As can be seen, the alignment interface 4000 comprises a left grid 4010a and a right grid 4010b, each of which comprises a matrix 4020 of twenty-five (25) light panels. An outer square 4030 comprises sixteen (16) of the light panels 4020. Each of the light panels 4020 of the outer square 4030 is backlit by a red colored light emitting diode (LED) 4035, shown in FIG. 10b. One of the light panels 4020 of the outer square 4030 is illuminated if the medical device is positioned greater than 3 mm from the target point associated with the target region. An inner square 4040 comprises eight (8) of the light panels 4020. Each of the light panels 4020 of the inner square 4040 is backlit by a yellow colored LED 4045, shown in FIG. 10b. One of light panels 4020 of the inner square 4040 is illuminated if the medical device is positioned greater than 0.25 mm from the target point associated with the target region, but less than 3 mm. A center light panel 4050 is backlight by a green colored LED 4055, shown in FIG. 10b, and is illuminated if the medical device is positioned within 0.25 mm from the target point associated with the target region.

FIG. 10b shows the circuit components of the alignment interface 4000. As can be seen, the LEDs 4035, 4045 and 4055 are surface mounted and are connected to a microcontroller 4060 and a voltage regulator 4070. A single shielded cable 4080 couples the alignment interface 4000 to the general purpose computing device 2000.

As shown in FIG. 10c, mesh copper shielding 4090 is used to back the LEDs 4035, 4045 and 4055 and a translucent lens 4100 is positioned adjacent to each of the LEDs 4035, 4045 and 4055. Solid copper shielding 4110 is used to back the microcontroller 4060 and voltage regulator 4070. As will be appreciated, copper shielding 4090 and 4110 is used to prevent the alignment interface 4000 from introducing noise into the MR imaging scanner 5000 and to prevent the coils of the MR imaging scanner 5000 from interfering with the operation of the alignment interface 4000. All components of the alignment interface 4000 are non-magnetic.

During operation, the device guiding apparatus 3000 is positioned within the bore of the MR imaging scanner 5000 and adjacent to a target region. For example, in the event the target region is a patient's prostate, the device guiding apparatus 3000 is positioned in between the patient's legs. The configuration of the device guiding apparatus 3000 allows for the bulk of the components of the device guiding apparatus 3000 to be positioned above the patient's legs, allowing the physician to have a direct line-of-site of the rear needle template 3420c from outside of the bore of the MR imaging scanner 5000.

As mentioned previously, the device guiding apparatus 3000 is operable in two modes: target only mode and target and entry mode.

During operation in the target only mode, the physician selects a target point associated with the target region and the alignment interface 4000 instructs the physician how to adjust the needle trajectory such that it will contact the target point. A forward kinematics solution is used to compare the target point with the intersection of the needle with an axial plane that contains the target point.

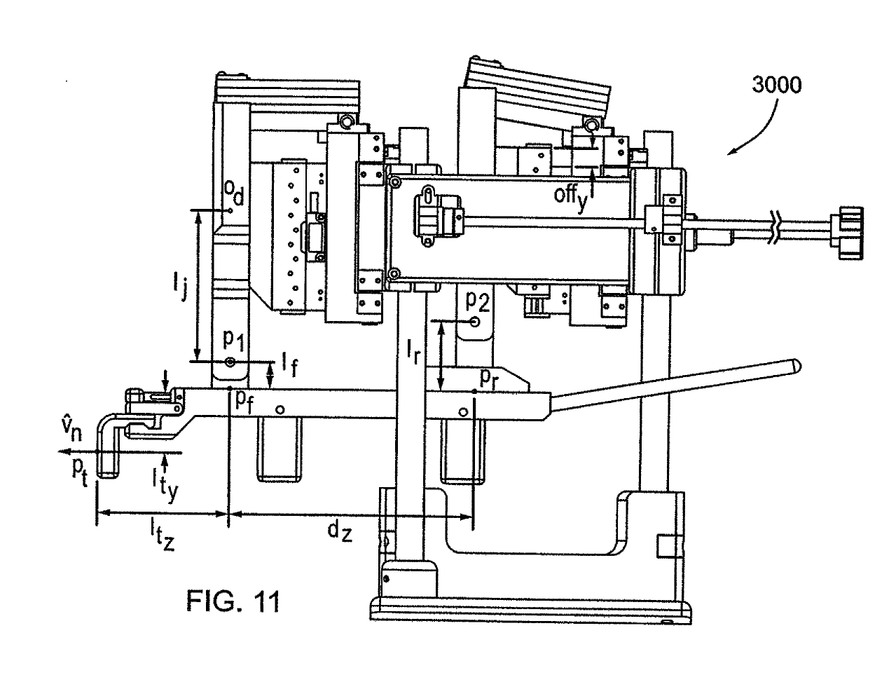

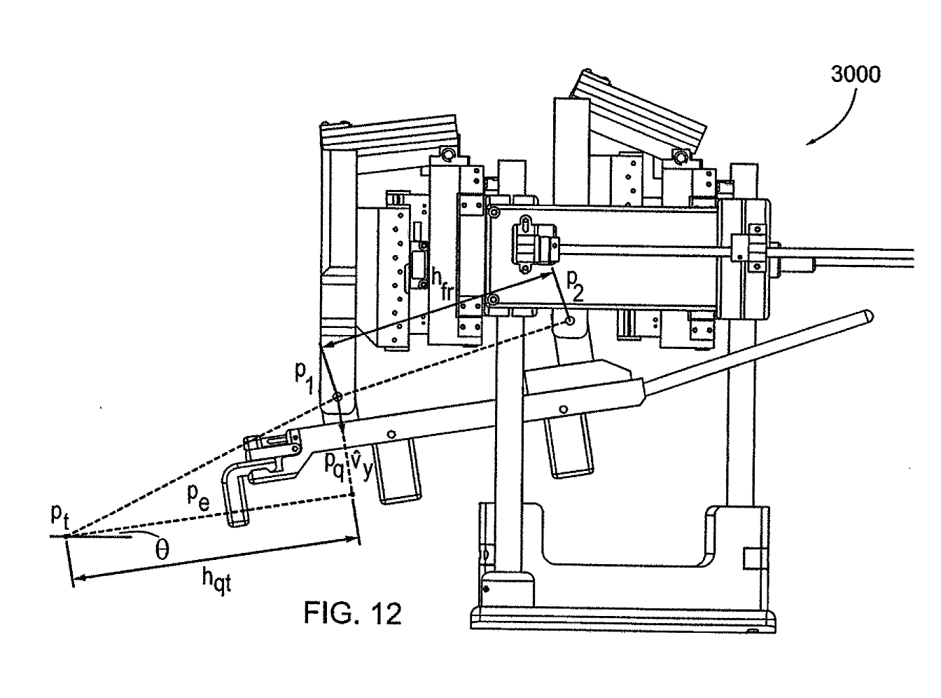

FIGS. 11 and 12 illustrate the device guiding apparatus 3000 showing constants, the device guiding apparatus 3000 origin O_{d}, and the needle trajectory defined by point pₜ and vector \hat{v}ₙ used to solve the forward kinematics solution.

To solve the forward kinematics solution, the (x,y) coordinates of linear motion assemblies 3200a and 3200b are measured by encoders S1 to S4 as coordinates (e₁ₓ, e_{1y}) and (e₂ₓ, e_{2y}). Intermediate variables are defined, as shown in FIGS. 11 and 12, and are calculated according to the following equations:

δ_{y}e_{2y}−e_{1y}−off_{y}, (1)

where δ_{y} is the position of the linear motion assembly 3200b relative to the front in the y-direction,

δ_{fr}=lᵣ−l_{f}, (2)

where δ_{fr} is a link constant that is equal to the difference between lengths of the spherical joints 3330 and 3340,

h_{fr}=\sqrt{d_{z}²+(δ_{fr}−δ_{y})²}, (3)

where h_{fr} is the direct distance between points p₁ and p₂,

$$θ = {tan}^{- 1} ( \frac{{δ}_{fr} - {δ}_{y}}{{d}_{z}} ) - {sin}^{- 1} ( \frac{{δ}_{fr}}{{h}_{yz}} ) ,$$

where θ is the angle the needle trajectory makes with the horizontal, and

$${\hat{v}}_{y} = [ \begin{matrix}0 \\ cos ( θ ) \\ - sin ( θ )\end{matrix} ] . \qquad ( 5 )$$

where \hat{v}_{y} is a unit vector that represents the orientation of the front gimbal.
Points pᵣ and p_{f} are defined as:

$$\begin{matrix}{p}_{r} = [ \begin{matrix}- {e}_{2 x} \\ {e}_{2 y} - {δ}_{fr} + {l}_{r} cos ( θ ) + {off}_{y} + {l}_{j} \\ - {l}_{r} sin ( θ ) - {d}_{z}\end{matrix} ] , \\ and & ( 6 ) \\ {p}_{f} = [ \begin{matrix}- {e}_{1 x} \\ {e}_{1 y} + {l}_{f} cos ( θ ) + {l}_{j} \\ - {l}_{f} sin ( θ )\end{matrix} ] . & ( 7 )\end{matrix}$$

The needle trajectory (point pₜ and needle vector \hat{v}ₙ) is calculated as:

$$\begin{matrix} {\hat{v}}_{n} = \frac{{p}_{f} - {p}_{r}}{\vert {p}_{f} - {p}_{r} \vert} , \\ and & ( 8 ) \\ {p}_{t} = {p}_{f} + \text{?} {\hat{v}}_{y} + \text{?} {\hat{v}}_{n} . \\ \text{?} \text{indicates text missing or illegible when filed} & ( 9 )\end{matrix}$$

Only the left grid 4010a of the alignment interface 4000 is used and helps the physician align the medical device (which in this embodiment is a needle) with the target point. To determine which one of the light panels 4020 is to be illuminated, the target point is compared to the intersection of the needle trajectory with an axial plane that contains the target point, as determined using the forward kinematics equations. If the x-component of the difference is greater than 3 mm, one of the light panels 4020 associated with one of the exterior columns of the outer square 4030 is illuminated. If the x-component of the difference is between 0.25 mm and 3 mm, one of the light panels 4020 associated with one of the exterior columns of the inner square 4040 is illuminated. If the x-component of the difference is less than 0.25 mm, the center light panel 4050 is illuminated. Similarly, if the y-component of the difference is greater than 3 mm, one of the light panels 4020 associated with one of the exterior rows of the outer square 4030 is illuminated. If the y-component of the difference is between 0.25 mm and 3 mm, one of the light panels 4020 associated with one of the exterior rows of the inner square 4040 is illuminated. If the y-component of the difference is less than 0.25 mm, the center light panel 4050 is illuminated.

During operation in the target and entry mode, the physician selects a target point and entry point and the alignment interface 4000 instructs the physician how to adjust the needle trajectory using all four degrees-of-freedom such that it will enter the patient at the entry point and will contact the target point. A reverse kinematics solution is used to calculate the position of each linear motion assembly 3200a and 3200b required for the device guiding apparatus 3000 to be aligned with a particular needle orientation.

FIGS. 11 and 12 illustrate the device guiding apparatus 3000 showing constants, the device guiding apparatus 3000 origin O_{d}, and the needle trajectory defined by point pₜ and vector \hat{v}ₙ used to solve the reverse kinematics solution. Points pₜ and pₑ are defined using the MR imaging scanner 5000. Point pₜ is the target point associated with the target region that the needle is to contact. Point pₑ is the entry point on the patients' skin that ensures the needle trajectory will not contact any critical structures within the patient's body while moving towards point pₜ. Intermediate variables are defined and are calculated according to the following equations:

$${\hat{v}}_{n} = \frac{{p}_{t} - {p}_{e}}{\vert {p}_{t} - {p}_{e} \vert} , \qquad ( 10 )$$

where \hat{v}ₙ is the needle vector,

$$\begin{matrix} θ = {sin}^{- 1} ( {\hat{v}}_{{n}_{y}} / \sqrt{{\hat{v}}_{{n}_{y}}^{2} + {\hat{v}}_{{n}_{z}}^{2}} ) , \\ and & ( 11 ) \\ {h}_{gt} = \frac{{p}_{{t}_{z}}}{cos ( θ )} + \text{?} tan ( θ ) , \\ \text{?} \text{indicates text missing or illegible when filed} & ( 12 )\end{matrix}$$

where h_{qt} is the base of the triangle connecting pᵢ and p₁ in FIG. 12. Next, p₁ is defined as:

$${p}_{1} = \text{?} - [ \begin{matrix}\text{?} cos ( θ ) {i}_{{z}_{x}} / \text{?} \\ \text{?} sin ( θ ) + \text{?} cos ( θ ) \\ \text{?} cos ( θ ) - \text{?} sin ( θ )\end{matrix} ] , \\ \text{?} \text{indicates text missing or illegible when filed} \qquad$$

and the linear motion offsets:

δ_{y}=δ_{fr}[1−1/cos(θ)]−d_{z} tan(θ),

and

δₓ=\hat{v}ₙ_{ₓ}[d_{z}+δ_{fr} sin(θ)]/\hat{v}ₙ_{_{z}}.

The desired (x,y) coordinates of linear motion assemblies 3200a and 3200b set as coordinates (e₁ₓ, e_{1y}) and (e₂ₓ, e_{2y}) are calculated as:

e₁ₓ=p₁_{ₓ}, (13)

e_{1y}=p₁_{_{y}}lⱼ, (14)

e₂ₓ=e₁ₓ+δₓ, (15)

and

e_{2y}=e_{1y}+δ_{y}−off_{y}. (16)

Both the left grid 4010a and right grid 4010b of the alignment interface 4000 are used. The left grid 4010a is used to help the physician align the linear motion assembly 3200a and the right grid is used to help the physician align the linear motion assembly 3200b. To determine which one of the light panels 4020 is to be illuminated, the difference between the position of each linear motion assembly 3200a and 3200b and the target position as determined using the reverse kinematics equations is used. The same x-component and y-component rules used during operation in the target only mode are used during operation in the target and entry mode to determine which one of the light panels 4020 is illuminated.

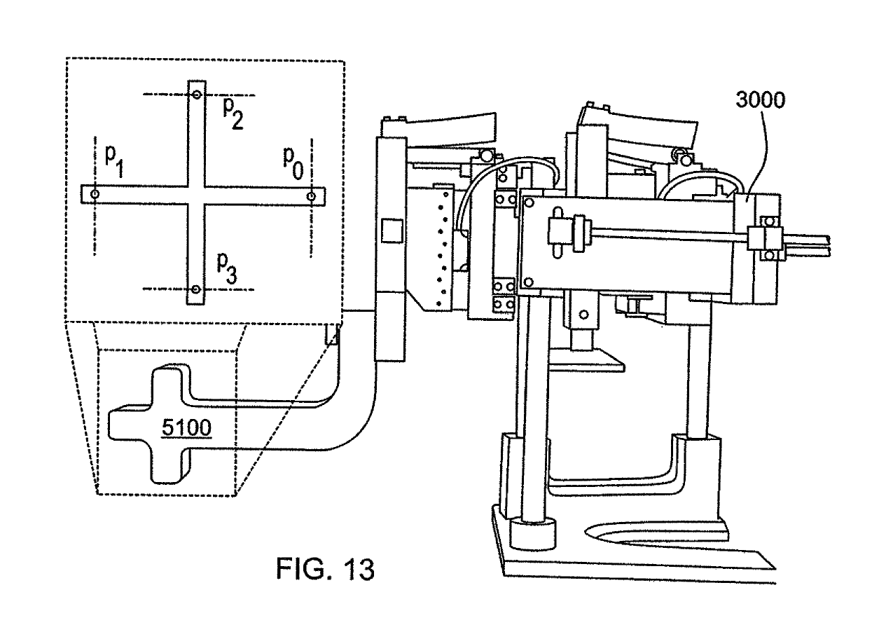

As will be appreciated, each time the device guiding apparatus 3000 is used, it is placed within the bore of the MR imaging scanner 5000. Thus, the device guiding apparatus 3000 coordinate system must be determined with respect to the MR imaging scanner 5000 at the beginning of each procedure to ensure accurate guidance. In this embodiment, a detachable fiducial MR-visible component 5100 is used and is shown in FIG. 13. The detachable component 5100 comprises two perpendicular drilled holes in the shape of a plus-sign “+”. The drilled holes are filled with an aqueous solution of 1% gadolinium by volume (Magnevist, 469 mg/ml). The detachable component 5100 is embedded within a plastic component and is mounted to the device guiding apparatus 3000. An exemplary sagittal MR image 5100′ of the detachable component 5100 is also shown in FIG. 13. Four points (p₀, p₁, p₂ and p₃) are identified on the MR image 5100′ and must be localized for registration. Dashed lines have been superimposed on the MR image 5100′ and indicate the image planes in which each point is localized. The points p₀ and p₁ are localized in axial images, and points p₂ and p₃ are localized in coronal images.

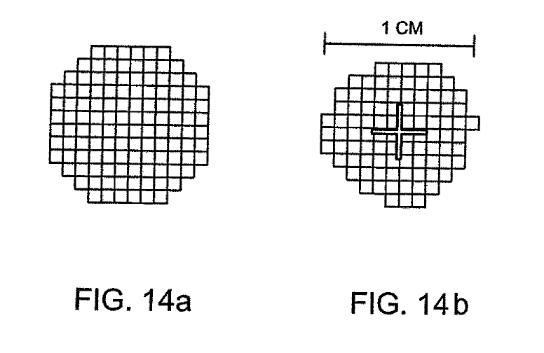

The captured MR images are filtered to reduce noise using a circular averaging filter having a radius of 2 pixels, and then thresholded. An exemplary fiducial image is shown in FIG. 14a. FIG. 14b shows the fiducial image of FIG. 14a once it has been filtered and thresholded. Since the size of each fiducial tube is known, the threshold value is chosen such that the total area of the thresholded image is equal to the known area of a section of a fiducial tube. Fiducial localization is then performed by the general purpose computing device 2000 to compute an intensity-weighted centroid of the filtered, thresholded image according to the following equation:

$$\text{?} = \frac{{\sum }_{j = 1}^{m} {\sum }_{k = 1}^{n} I ( j , k ) \text{?} ( j , k )}{{\sum }_{j = 1}^{m} {\sum }_{k = 1}^{n} I ( j , k )} \\ \text{?} \text{indicates text missing or illegible when filed} \qquad ( 20 )$$

where xᵢ(j, k) is the i^{th} coordinate of the pixel at index (j, k), I(j,k) is the corresponding pixel intensity, and x̅ᵢ is the i^{th} coordinate of the centroid of the image of size m×n.

Sensitivity of fiducial localization to main field inhomogeneity is reduced by measuring coordinates in the phase encode direction of each image. Accordingly, two sets of images of each fiducial are acquired, with the phase encoder direction swapped in each acquisition. Since the axes of the device guiding apparatus 3000 are generally aligned with those of the MR imaging scanner 5000, error in pose estimation of the fiducial arrangement due to slice-select error is minimal. The four points are used to compute the unit vectors in the direction of each of the device guiding apparatus' axes, in MR coordinates, as:

$${\hat{z}}_{d} = \frac{{p}_{1} - {p}_{0}}{\vert {p}_{1} - {p}_{0} \vert} , \\ {\hat{x}}_{d} = \frac{- ( {p}_{1} - {p}_{0} ) \times ( {p}_{3} - {p}_{2} )}{\vert ( {p}_{1} - {p}_{0} ) \times ( {p}_{3} - {p}_{2} ) \vert} , \\ and \\ {\hat{y}}_{d} = {\hat{z}}_{d} \times {\hat{x}}_{d} , \qquad ( 21 )$$

and the origin as the closes point to the line that passes through p₀ and p₁, and that which passes through p₂ and p₃. As such, points in the device guiding apparatus 3000 are converted to the MR imaging scanner's 5000 coordinate system using:

(pₘᵣ)ᵢ=(p_{d})₁(\hat{x}_{d})ᵢ+(p_{d})₂(ŷ_{d})ᵢ+(p_{d})₃(\hat{z}_{d})ᵢ+(o_{d})ᵢ (22)

where p_{d} is a point in the device guiding apparatus 3000, and pₘᵣ is the point in the coordinate system of the MR imaging scanner 5000. Points in the coordinate system of the MR imaging scanner 5000 can be converted to coordinates in the device guiding apparatus 3000 by solving the linear system:

$$[ \begin{matrix}{( {\hat{x}}_{d} )}_{1} & {( {\hat{y}}_{d} )}_{1} & {( {\hat{z}}_{d} )}_{1} \\ {( {\hat{x}}_{d} )}_{2} & {( {\hat{y}}_{d} )}_{2} & {( {\hat{z}}_{d} )}_{2} \\ {( {\hat{x}}_{d} )}_{3} & {( {\hat{y}}_{d} )}_{3} & {( {\hat{z}}_{d} )}_{3}\end{matrix} ] [ \begin{matrix}{( {p}_{d} )}_{1} \\ {( {p}_{d} )}_{2} \\ {( {p}_{d} )}_{3}\end{matrix} ] = [ \begin{matrix}{( {p}_{mr} )}_{1} - {( {o}_{d} )}_{1} \\ {( {p}_{mr} )}_{2} - {( {o}_{d} )}_{2} \\ {( {p}_{mr} )}_{3} - {( {o}_{d} )}_{3}\end{matrix} ] . \qquad ( 23 )$$

The registration fiducials are placed at the MR imaging scanner's 5000 isocenter, scanned before the patient is positioned, and removed from the device guiding apparatus 3000 before the patient arrives, thereby reducing the amount of time the patient must be anesthetized.

Although in embodiments described above the user is described as being a physician, those skilled in the art will appreciate that other types of users may use the system.

Although in embodiments described above the orientation of the medical device is adjusted manually via a manipulator arm connected to an adjustment handle, those skilled in the art will appreciate that the orientation of the medical device may be adjusted automatically. In this embodiment, the linear motion assemblies are adjusted through a motor assembly comprising one or more motors.

Although in embodiments described above the medical device is described as being a needle, those skilled in the art will appreciate that other medical devices may be used such as for example a catheter.

Although in embodiments described above the target region is described as being the prostate, those skilled in the art will appreciate that other target regions may be targeted with the system such as for example the brain, the cervix, etc.

Although in embodiments described above the device guiding apparatus is described as utilizing sensors in the form of magnetic rotary encoders, those skilled in the art will appreciate that other instruments may be used to determine position such as for example optical encoders, incremental or absolute encoders, linear encoders, optical tracking systems using a set of stereo cameras a reflective markers, mechanical scales (Vernier scales, etc.), a tracking system using one or more imaging sensors by imaging registration fiducials in real-time as the device is being moved, a stepper motor system wherein, assuming no slip, the number of steps a motor has been directed are used to determine the position of the medical device, etc.

Although in embodiments described above the device guiding apparatus is used in conjunction with a MR imaging scanner, it will be appreciate that device guiding apparatus may be used in conjunction with other types of imaging scanners. For example, imaging scanners such as for example a computed tomography (CT) scanning system, a positron emission tomography (PET) scanning system, a single-photon emission computed tomography (SPECT) scanning system, an ultrasound scanning system, etc.

Although in embodiments described above the various components of the device guiding apparatus are made of plastic materials, those skilled in the art will appreciate that the types of materials used for the various components of the system is dependent on the type of imaging scanner used in conjunction with the system.

One skilled in the art will appreciate that the device guiding apparatus may be used for imaging humans and/or animals.

Although embodiments are described above with reference to the accompanying drawings, those skilled in the art will appreciate that variations and modifications may be made without departing from the scope thereof as defined by the appended claims.

### REFERENCES

1. O. Raz, M. A. Haider, S. R. H. Davidson, U. Lindner, E. Hlasny, R. Weersink, M. R. Gertner, W. Kucharcyzk, S. A. McCluskey and J. Trachtenberg, “Real-time magnetic resonance imaging-guided focal laser therapy in patients with low-risk prostate cancer,” Eur. Urol. 58, 173-177 (2010).
2. G. S. Fischer, I. Iordachita, C. Csoma, J. Tokuda, S. P. DiMaio, C. M. Tempany, N. Hata and G. Fichtinger, “MRI-compatible pneumatic robot for transperineal prostate needle placement,” IEEE Transactions on Mechatronics 13, 295-305 (2008).
3. A. A. Goldenberg, J. Trachtenberg, Y. Yi, R. Weersink, M. S. Sussman, M. Haider, L. Ma and W. Kucharezyk, “Robot-assisted MRI-guided prostatic interventions,” Robotica 28, 215 (2010).
4. A. Krieger, C. Csoma, Iordachital, II, P. Guion, A. K. Singh, G. Fichtinger and L. L. Whitcomb, “Design and preliminary accuracy studies of an MRI-guided transrectal prostate intervention system,” Med Image Comput Comput Assist Interv 10, 59-67 (2007).
5. A. Krieger, I. Iordachita, S. E. Song, N. B. Cho, P. Guion, G. Fichtinger and L. L. Whitcomb, “Development and Preliminary Evaluation of an Actuated MRI-Compatible Robotic Device for MRI-Guided Prostate Intervention,” IEEE Int. Conf. Robot., 1066-1073 (2010).
6. M. G. Schouten, J. Ansems, W. K. Renema, D. Bosboom, T. W. Scheenen and J. J. Futterer, “The accuracy and safety aspects of a novel robotic needle guide manipulator to perform transrectal prostate biopsies,” Med. Phys. 37, 4744-4750 (2010).
7. D. Stoianovici, D. Song, D. Petrisor, D. Ursu, D. Mazilu, M. Muntener, M. Schar and A. Patriclu, ““MRI Stealth” robot for prostate interventions,” Minimally Invasive Therapy & Allied Technologies 16, 241-248 (2007).
8. S. Zangos, C. Herzog, K. Eichler, R. Hammerstingl, A. Lukoschek, S. Guthmann, B. Gutmann, U. J. Schoepf, P. Costello and T. J. Vogl, “MR-compatible assistance system for punction in a high-field system: device and feasibility of transgluteal biopsies of the prostate gland,” Eur. Radial. 17, 1118-1124 (2007).
9. G. Fischer, A. Krieger, I. Iordachita, C. Csoma, L. Whitcomb and G. Fichtinger, “MRI compatibility of robot actuation techniques—a comparative study,” Medical Image Computing and Computer-Assisted Intervention—MICCAI 2008, 509-517 (2008).

The relevant portions of the references identified in the specification are incorporated herein by reference.

What is claimed is:

1. A device guiding apparatus, comprising:
support framework;
a counterbalance supported by the support framework at a position above a surface on which the support framework rests; and
a manipulation assembly supported by the counterbalance, the manipulation assembly comprising at least one support assembly for supporting a medical device at a position intermediate the counterbalance and the surface such that a user has a direct line-of-site of the at least one support assembly.

2. The device guiding apparatus of claim 1, wherein the support framework, the manipulation assembly, the at least one support assembly and the counterbalance are made of non-magnetic materials.

3. The device guiding apparatus of claim 2, wherein the device guiding apparatus is positionable within a bore of a magnetic resonance (MR) imaging scanner.

4. The device guiding apparatus of claim 1, wherein the medical device is a needle.

5. The device guiding apparatus of claim 1, comprising a sensor arrangement configured to obtain sensor data and processing structure for processing the sensor data to determine the trajectory of the medical device.

6. The device guiding apparatus of claim 5, comprising an alignment interface providing feedback to the user for adjusting the trajectory of the medical device.

7. The device guiding apparatus of claim 1, wherein the support framework is configured to provide working space for the user.

8. The device guiding apparatus of claim 1, comprising:
at least one extension arm connected at a first end to the counterbalance and at a second end to the manipulation assembly; and
at least one linear motion assembly connected to the at least one extension arm at a position intermediate the first and second end, the at least one linear motion assembly allowing for manipulation of the trajectory of the medical device.

9. The device guiding apparatus of claim 8, comprising at least one locking mechanism supported by the support framework, the at least one locking mechanism configured to prevent movement of the at least one linear motion assembly when in a first position and configured to permit movement of the at least one linear motion assembly when in a second position.

10. A device guiding apparatus, comprising:
support framework;
a counterbalance supported by the support framework at a position above a surface on which the support framework rests; and
a manipulation assembly supported by the counterbalance, the manipulation assembly comprising at least one support assembly for supporting a medical device at a position intermediate the counterbalance and the surface such that a user has a direct line-of-site of the at least one support assembly;
a sensor arrangement configured to obtain sensor data; and
processing structure configured to:
receive sensor data from the sensor arrangement;
process the received sensor data to determine the trajectory of the medical device;
calculate a point of intersection with a target region based on the trajectory of the medical device;
calculate a difference between the point of intersection and a target point associated with the target region; and
provide feedback to the user to guide the medical device to the target point based on said calculated difference.

11. The device guiding apparatus of claim 10, wherein the support framework, the manipulation assembly, the at least one support assembly, the counterbalance and the sensor arrangement are made of non-magnetic materials.

12. The device guiding apparatus of claim 11, wherein the device guiding apparatus is positionable within a bore of a magnetic resonance (MR) imaging scanner.

13. The device guiding apparatus of claim 10, wherein the medical device is a needle.

14. The device guiding apparatus of claim 10, wherein the sensor arrangement comprises a plurality of magnetic rotary encoders.

15. The device guiding apparatus of claim 10, wherein the processing structure provides feedback to the user via an alignment interface.

16. A method for providing feedback to a user guiding a medical device to a target region, the method comprising:
receiving sensor data from a sensor arrangement;
processing the received sensor data to determine the trajectory of the medical device;
calculating a point of intersection with a target region based on the trajectory of the medical device;
calculating a difference between the point of intersection and a target point associated with the target region; and
providing feedback to the user based on the calculated difference between the point of intersection and the target point.

17. The method of claim 16, further comprising:
adjusting the trajectory of the medical device based on the calculated difference between the point of intersection and the target point.

18. The method of claim 16, further comprising:
calculating a difference between the point of intersection and an entry point associated with the target point; and
providing feedback to the user based on the calculated difference between the point of intersection and the entry point.

19. The method of claim 18, further comprising:
adjusting the trajectory of the medical device based on the calculated difference between the point of intersection and the target point and based on the calculated difference between the point of intersection and the entry point.

20. A non-transitory computer readable medium having stored thereon a computer program comprising computer readable instructions for execution by a computer to perform a method of providing feedback to a user guiding a medical device to a target region, the method comprising:
receiving sensor data from a sensor arrangement;
processing the received sensor data to determine the trajectory of the medical device;
calculating a point of intersection with a target region based on the trajectory of the medical device;
calculating a difference between the point of intersection and an actual target point associated with the target region; and
providing feedback to the user based on the calculated difference between the estimated target point and the actual target point.
